(19) United States
(12) Patent Application Publication
(10) Pub. No.: US 2025/0253038 A1
(43) Pub. Date: Aug. 7, 2025

(54) System, Method and Computer Program Product for Monitoring Diabetics

(71) Applicant: Celloscope Ltd., Tel Aviv (IL)

(72) Inventors: Yuval NAVEH, Haifa (IL); Tamir ALOUSH, Kiryat Ono (IL); Levi TEITZ, Tel Aviv (IL); Shahar DAVIDSON, Rehovot (IL); Tomer SHUSSMAN, Kfar Saba (IL)

(21) Appl. No.: 19/049,400

(22) Filed: Feb. 10, 2025

Related U.S. Application Data
(63) Continuation-in-part of application No. 18/939,288, filed on Nov. 6, 2024.
(63) Continuation-in-part of application No. 18/975,890, filed on Dec. 10, 2024.
(60) Provisional application No. 63/596,479, filed on Nov. 6, 2023.
(60) Provisional application No. 63/612,587, filed on Dec. 20, 2023.
(60) Provisional application No. 63/557,740, filed on Feb. 26, 2024.
(60) Provisional application No. 63/557,747, filed on Feb. 26, 2024.
(60) Provisional application No. 63/557,753, filed on Feb. 26, 2024.
(60) Provisional application No. 63/557,757, filed on Feb. 26, 2024.
(60) Provisional application No. 63/557,762, filed on Feb. 26, 2024.

(51) Int. Cl. G16H 40/20; G06T 7/00; G06T 7/90; G16H 10/60; G16H 40/67; H04W 4/02

(52) U.S. Cl. CPC G16H 40/20; G06T 7/0016; G06T 7/90; G16H 10/60; G16H 40/67; H04W 4/025; G06T 2207/30196

(57) ABSTRACT

A health management system comprising a personnel allocation processor for allocating personnel to conduct home or clinic sessions e.g. with diabetes patients who may be known to the system; and/or a gait analysis processor receiving IMU data e.g. from cellphones which may be worn by the diabetes patients and/or providing the personnel allocation processor with indications of a subset of the diabetes patients whose IMU data indicates new deterioration. Typically, allocation of personnel by the personnel allocation processor at least partly favors diabetes patients falling within the subset over diabetes patients who do not fall within the subset. The personnel allocation processor may comprise doctors' office software for managing appointments, which may have priority logic accepting prioritization e.g. of at least one patient in the subset over at least one patient not in the subset, via a machine interface.

### CROSS-REFERENCE TO RELATED APPLICATIONS

The present application is a continuation-in-part of application Ser. No. 18/939,288, filed Nov. 6, 2024, and of application Ser. No. 18/975,890, filed Dec. 10, 2024, the entire contents of each of which being hereby fully incorporated herein by reference.

The present application further claims benefit, directly or indirectly, of the following provisional applications, the entire contents of each of which being fully incorporated herein by reference: Application No. 63/557,740, filed Feb. 26, 2024; Application No. 63/557,747 (Mobility Assessment in Patients with Diabetes), filed Feb. 26, 2024; Application No. 63/557,753, filed Feb. 26, 2024; Application No. 63/557,757, filed Feb. 26, 2024, Application No. 63/557,762, filed Feb. 26, 2024; Application No. 63/596,479, filed Nov. 6, 2023; and Application No. 63/612,587, filed Dec. 20, 2023.

### FIELD OF THIS DISCLOSURE

The present invention relates generally to computerized analysis of motion, and more particularly to computerized analysis of human motion which receives sensor outputs borne by a human, typically in real time.

### BACKGROUND FOR THIS DISCLOSURE

Diabetes management remains a significant problem despite all efforts to tackle the problem. Wikipedia's entry on “Diabetes management” explains inter alia that:

“Prolonged and elevated levels of glucose in the blood, . . . over time, result in serious diabetic complications . . . and sometimes even death. There is currently no way of testing for susceptibility to complications. Diabetics are therefore recommended to check their blood sugar levels either daily or every few days. There is also diabetes management software available from blood testing manufacturers which can display results and trends over time.

. . . The prevalence of medication nonadherence is high among patients with chronic conditions, such as diabetes, and nonadherence is associated with public health issues and higher health care costs . . . . Being able to detect cost-related nonadherence . . . can lead to strategies to assist patients with problems paying for their medications . . . . Interventions to improve adherence can achieve reductions in diabetes morbidity and mortality, as well as significant cost savings to the health care system. Smartphone apps have been found to improve self-management and health outcomes in people with diabetes through functions such as specific reminder alarms, while working with mental health professionals has also been found to help people with diabetes develop the skills to manage their medications and challenges of self-management effectively . . . .

As self-management of diabetes typically involves lifestyle modifications, adherence may pose a significant self-management burden on many individuals . . . there has been evidence suggesting the self-management of diabetes is negatively affected by diabetes-related distress and depression . . . . To this end, treatment programs such as the Cognitive Behavioural Therapy-Adherence and Depression program . . . aim to enhance self-efficacy and improve diabetes-related distress and one's overall quality of life”.

OneStep is an FDA-listed medical app, downloadable from GooglePlay, that uses smartphone motion sensors to provide immediate, clinically-validated feedback on gait inter alia.

The disclosures of all publications and patent documents mentioned above and elsewhere in the specification, and of the publications and patent documents cited therein directly or indirectly, are hereby incorporated herein by reference in their entirety. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

### SUMMARY OF CERTAIN EMBODIMENTS

Certain embodiments of the present invention seek to provide circuitry typically comprising at least one processor in communication with at least one memory, with instructions stored in such memory executed by the processor to provide functionalities which are described herein in detail. Any functionality described herein may be firmware-implemented or processor-implemented, as appropriate.

Certain embodiments seek to provide a cell app or web service including a patient interface and/or caregiver interface and/or health-providing organization interface, configured to lighten the burden of managing diabetes, a complicated chronic disease, which falls on all of these 3 stakeholders, including use of gait analysis according to any embodiments herein to facilitate at least one and preferably multiple aspects of the diabetic treatment cycle of end-users e.g. from screening for diabetics using gait analysis, to diagnosing diabetics, to treating early-stage diabetics, to treating late-stage diabetics including complications, to treating amputees and/or end-of-life diabetics, to recording death of the end-user aka patient. Typically this includes use of gait analysis to identify patients who have passed from one stage to another (and responsively, initiating a system response that is standard-of-care for the new stage) or may be passing from one stage to another (and responsively, alerting a clinician to confirm or refute this) or are at risk of passing to the next stage (and responsively, initiating measures that are known to reduce this risk and/or increasing monitoring frequency to more quickly detect that the user has passed to the next stage.

Certain embodiments seek to repeatedly identify a subset of registered end-users, which may be marked as known diabetes patients, with “new deterioration”. The system may monitor various gait parameters of at least some registered end-users (typically either including or consisting solely of those end-users known to be diabetes patients), e.g. cadence and/or step length and/or any gait parameter/s mentioned herein. Patients having, say, at least one gait parameter which has worsened by 20% or at least 2 gait parameters which have worsened by 10% or at least 4 gait parameters which have worsened by 5% may be considered to have a “new deterioration”. It is appreciated that this logical criterion is merely an example; any suitable criterion may be employed and may be machine-learned as the system matures e.g. by training a classifier to identify new deteriorations, on training data including historical imu data or gait parameters of end-users later diagnosed in clinic as having new deterioration/s and also including historical imu data or gait parameters of end-users later seen in clinic and for whom new deterioration/s were ruled out.

Certain embodiments seek to process imu data to predict or estimate typically time-stamped clinical states of patients e.g. standard outcomes (estimated scores), or presence/absence of particular diagnoses such as a foot ulcer and/or Charcot Foot.

Embodiments herein described in the context of diabetes, by way of example, are also suited to other disease contexts, including other chronic diseases, including predicting standard outcomes which are used in treating other disease contexts as opposed to standard outcomes useful for treating diabetes which are mentioned by way of example herein.

Embodiments include:
Embodiment 1. A health management system comprising: a personnel allocation processor for allocating personnel to conduct home or clinic sessions with diabetes patients known to the system; and/or a gait analysis processor typically configured for receiving IMU data from cellphones worn by the diabetes patients and typically providing the personnel allocation processor with indications of a subset of the diabetes patients whose IMU data indicates new deterioration; and wherein typically allocation of personnel by the personnel allocation processor at least partly favors diabetes patients falling within the subset over diabetes patients who do not fall within the subset; wherein the personnel allocation processor may comprise doctors' office software for managing appointments, such software having priority logic accepting prioritization via a machine interface.
Embodiment 2. A health management method comprising: using gait analysis of at least one end-user's motor behavior over a time period preceding or culminating at time-point T e.g. to generate at least one estimate of at least one standard outcome at time T, for the at least one end-user; and/or automatically adjusting healthcare for at least one end-user after time-point T, based at least on the estimate.

Healthcare adjustment may include providing or scheduling any care action described herein such as a treatment or such as an appointment to see a clinician or such as administration of a test.
Embodiment 3. A system according to any preceding embodiment wherein prioritization may comprise patients within the subset being defined as urgent and at least patients not falling within the subset being defined as routine rather than urgent.
Embodiment 4. A system according to any preceding embodiment wherein the system contacts patients within the subset to request and upload an image of the patient's foot, and wherein the image is automatically image-processed to identify discoloration each time the new deterioration suggests an increase in risk of gangrene.
Embodiment 5. A system according to any preceding embodiment wherein the system contacts patients within the subset to request and upload an image of the patient's foot, and wherein the image is automatically image-processed to identify changes in foot shape or alignment relative to previous images in the system and/or relative to population norms each time the new deterioration suggests an increase in risk that nerve damage has led to a weakening of bones in the foot.
Embodiment 6. A system according to any preceding embodiment wherein the gait analysis processor separately classifies peripheral artery disease which leads to leg pain, and peripheral artery disease which leads to numbness.
Embodiment 7. A system according to any preceding embodiment wherein the gait analysis processor is trained to predict at least one direct testing result, such as ankle brachial index test, or foot imaging tests evaluating arterial blockage, such as ultrasound or angiography.
Embodiment 8. A system according to any preceding embodiment wherein the gait analysis processor has a machine interface for diabetes patients' medical records, and wherein symptoms such as peripheral artery disease are automatically added to patients' medical records responsive to classification of a patient as suffering from the symptoms by the gait analysis processor with an option for human override and/or with an indication in the medical record that the symptom has been added to the medical record using machine intelligence.
Embodiment 9. A system according to any preceding embodiment wherein the gait analysis processor, responsive to identification of diabetic neuropathy by the system, is configured to schedule evaluations of neuropathic arthropathy, where each evaluation includes prompting the patient identified as having diabetic neuropathy to upload an image, and automatically processing the image to identify foot shape changes.
Embodiment 10. A computer program product, comprising a typically non-transitory computer-usable or -readable medium e.g. non-transitory computer-usable or -readable storage medium, having a computer readable program code embodied therein, said computer readable program code adapted to be executed to implement a health management method comprising: using gait analysis of at least one end-user's motor behavior e.g. over a time period preceding or culminating at time-point T to generate at least one estimate of typically at least one standard outcome at time T, for the at least one end-user; and/or automatically adjusting healthcare for at least one end-user after time-point T, e.g. based at least on said estimate.

Also provided, excluding signals, is a computer program comprising computer program code means for performing any of the methods shown and described herein when said program is run on at least one computer; and a computer program product, comprising a typically non-transitory computer-usable or -readable medium e.g. non-transitory computer-usable or -readable storage medium, typically tangible, having a computer readable program code embodied therein, said computer readable program code adapted to be executed to implement any or all of the methods shown and described herein. The operations in accordance with the teachings herein may be performed by at least one computer specially constructed for the desired purposes, or a general-purpose computer specially configured for the desired purpose by at least one computer program stored in a typically non-transitory computer-readable storage medium. The term “non-transitory” is used herein to exclude transitory, propagating signals or waves, but to otherwise include any volatile or non-volatile computer memory technology suitable to the application.

Any suitable processor/s, display and input means may be used to process, display, e.g., on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMS, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g., BLE) or wired (e.g., USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g., via a wired or cellular telephone network, or a computer network such as the Internet.

The apparatus of the present invention may include, according to certain embodiments of the invention, machine readable memory containing or otherwise storing, a program of instructions, which, when executed by the machine, implements all or any subset of the apparatus, methods, features, and functionalities of the invention shown and described herein. Alternatively, or in addition, the apparatus of the present invention may include, according to certain embodiments of the invention, a program as above which may be written in any conventional programming language, and optionally a machine for executing the program, such as but not limited to a general-purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities, e.g., within the computing system's registers and/or memories, and/or may be provided on-the-fly, into other data which may be similarly represented as physical quantities within the computing system's memories, registers or other such information storage, transmission or display devices or may be provided to external factors e.g. via a suitable data network. The term “computer” should be broadly construed to cover any kind of electronic device with data processing capabilities, including, by way of non-limiting example, personal computers, servers, embedded cores, computing systems, communication devices, processors (e.g., digital signal processors (DSPs), microcontrollers, field programmable gate arrays (FPGAs), application specific integrated circuits (ASICs), etc.) and other electronic computing devices. Any reference to a computer, controller, or processor, is intended to include one or more hardware devices, e.g., chips, which may be co-located or remote from one another. Any controller or processor may, for example, comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may be implemented in hardware, e.g., using one or more Application-Specific Integrated Circuits (ASICs) or Field-Programmable Gate Arrays (FPGAs) or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components, and alternatively may be the same structure. A statement that an element or feature may exist is intended to include (a) embodiments in which the element or feature exists; (b) embodiments in which the element or feature does not exist; and (c) embodiments in which the element or feature exist selectably, e.g., a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein and/or to perform functionalities described herein and/or to implement any engine, interface, or other system illustrated or described herein. Any suitable computerized data storage, e.g., computer memory, may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein, which may, for example, include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user's device, automated speech-to-text conversion tool, including a front-end interface portion thereof and back-end logic interacting therewith. Thus, the term user interface or “UI” as used herein includes also the underlying logic which controls the data presented to the user, e.g., by the system display, and receives and processes and/or provides to other modules herein, data entered by a user, e.g., using her or his workstation/device.

### BRIEF DESCRIPTION OF THE DRAWINGS

FIGS. 1-2 are simplified flowchart illustrations of methods provided in accordance with embodiments of the present invention which may be performed either separately or together. Arrows between operations may be implemented as APIs, and any suitable technology may be used for interconnecting functional components or modules described herein in a suitable sequence or order, e.g., via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML. According to one embodiment, one of the modules may share a secure API with another module. Communication between modules may comply with any customized protocol or customized query language, or may comply with any conventional query language or protocol.

Flows may include all or any subset of the illustrated operations, suitably ordered, e.g., as shown.

Computational, functional or logical components described and illustrated herein may be implemented in various forms, for example as hardware circuits, such as but not limited to custom VLSI circuits or gate arrays or programmable hardware devices such as but not limited to FPGAs, or as software program code stored on at least one tangible or intangible computer-readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences, such as but not limited to objects, procedures, functions, routines, and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology) or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device, and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer, or more generally by a suitable microprocessor, configured in accordance with methods shown and described herein, or any suitable subset, in any suitable order, of the operations included in such methods, or in accordance with methods known in the art

Any logical functionality described herein may be implemented as a real time application, if, and as appropriate, and which may employ any suitable architectural option, such as but not limited to FPGA, ASIC, or DSP, or any suitable combination thereof.

Any hardware component mentioned herein may in fact include either one or more hardware devices, e.g., chips, which may be co-located or remote from one another.

Any method described herein is intended to include, within the scope of the embodiments of the present invention, also any software or computer program performing all or any subset of the method's operations, including a mobile application, platform or operating system, e.g., as stored in a medium, as well as combining the computer program with a hardware device to perform all or any subset of the operations of the method.

Data may be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary, or off-line; which is of any type or level or amount or category of volatility, differentiation, mutability, accessibility, addressability, capacity, performance and energy use; and which is based on any suitable technologies such as semiconductor, magnetic, optical, paper, and others.

### DETAILED DESCRIPTION OF CERTAIN EMBODIMENTS

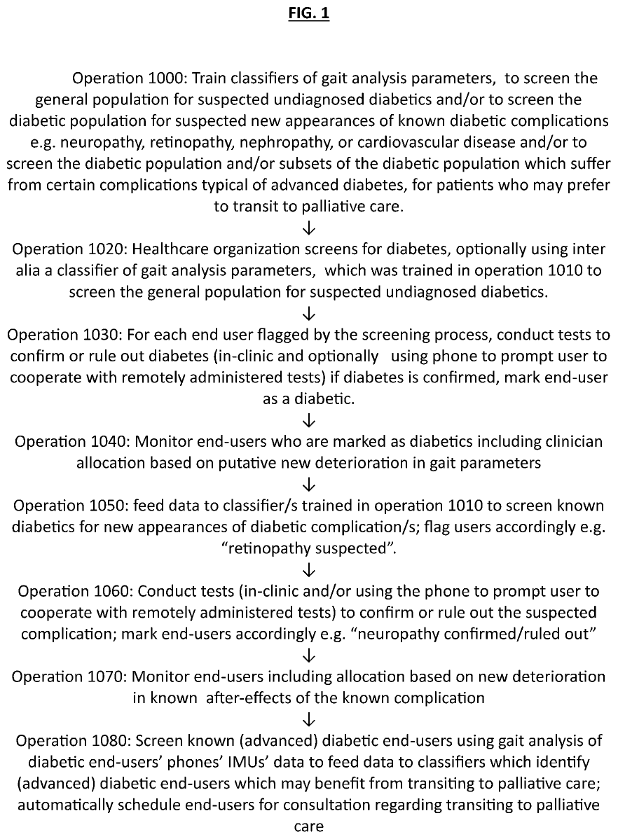

A method of operation for a system according to an embodiment herein may include all or any subset of the following operations in any suitable order, e.g. as shown in FIG. 1:

Operation 1000: Train classifiers of gait analysis parameters, to screen the general population for suspected undiagnosed diabetics and/or to screen the diabetic population for suspected new appearances of known diabetic complications e.g. neuropathy, retinopathy, nephropathy, or cardiovascular disease and/or to screen the diabetic population and/or subsets of the diabetic population which suffer from certain complications typical of advanced diabetes, for patients who may prefer to transit to palliative care.

For example, gait parameters during passive walks may be collected from a healthy population and also from a population of end-users diagnosed with a given complication e.g., retinopathy, typically say 50 people in each of the 2 groups, and, using this as training data, a classifier may be trained to predict to which group a given passive walk belongs, e.g. does the end-user, whose IMU (Inertial Measurement Unit) supplied the passive walk, suffer or not suffer from retinopathy. Herein, “passive” is intended to include IMU data collection from a user's phone's IMU, which does not require user cooperation each time the IMU data is collected and in fact the user may be unaware that IMU data is being collected e.g. because IMU data collection and subsequent analysis do not require that the user be prompted to provide any information or to perform any particular action. For example, the system typically does not require a user to be prompted to walk or to indicate whether or not s/he is walking. Instead, the disclosures of co-owned U.S. Ser. No. 18/975,890 entitled “Advanced Pedestrian Navigation Based on Inertial Gait Analysis and GPS Data” and/or of co-owned US20220111257 entitled “System . . . for Sensor-Based Enhancement of Physical Rehabilitation” (both of which are hereby incorporated herein by reference in their entirety) may be used to automatically classify a given time-interval as including gait or not e.g. by determining whether the user is ambulating during that interval. Then, IMU data may if desired to be collected only during such intervals which, as automatically determined, do include ambulation as opposed to intervals in which an end-user is stationary e.g.

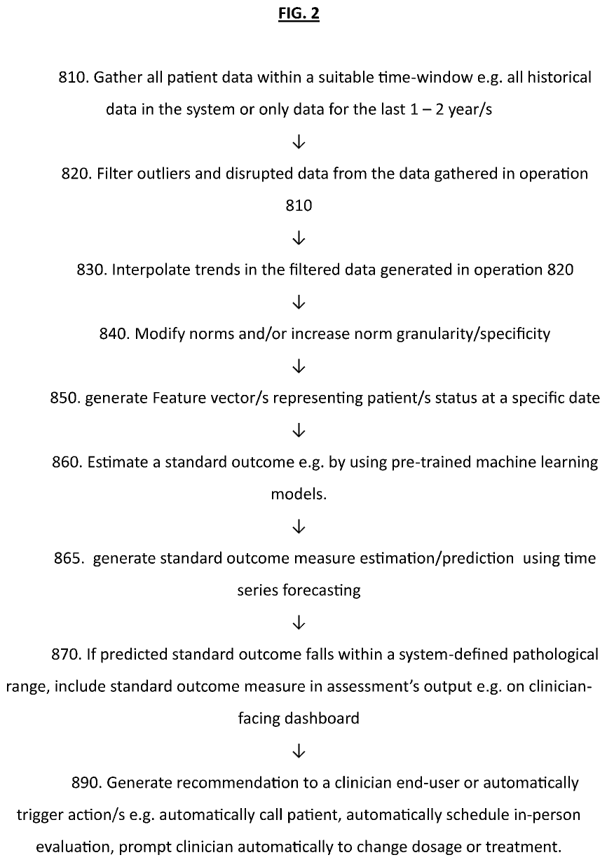

It is appreciated that neuropathy may be predicted using gait patterns. A standard outcome of neuropathic-type pain e.g. burning, tingling, or stabbing (typically of a certain level e.g. a given score on visual analog scale for pain and in a certain part of the body such as in the feet, legs, or hands) may be estimated e.g. using the method of FIG. 2 as described herein especially if some users in the system have actual clinical events of neuropathic-type pain which are already recorded in the system.

Alternatively or in addition a classifier may be trained that gets a measurement of gait using an IMU and detects pain level and position.

Nophropathy and/or cardiovascular disease may generate fatigue and/or weakness which the system may identify as a low score on a 6-minute walk test, relative to a patient's own norms, or relative to population norms.

Operation 1020: Healthcare organization e.g. HMO screens for diabetes, especially type 2 diabetes, e.g. using fasting blood sugar test, hemoglobin a1c test, oral glucose tolerance test (OGTT), random blood sugar test, or patient reports of diabetes symptoms, such as excessive thirst and/or excessive urination, and/or using a classifier of gait analysis parameters, which was trained in operation 1010 to screen the general population for suspected undiagnosed diabetics.

Operation 1030: For each end user flagged by the screening process, conduct tests to confirm or rule out diabetes (in-clinic and optionally using phone to prompt user to cooperate with remotely administered tests e.g. to prioritize between the flagged users to determine who should most urgently be tested); if diabetes is confirmed, mark end-user as a diabetic. Typically, if diabetes is ruled out, this datum is entered in the system, time-stamped, to mark the end-user in question.

Operation 1040: Monitor end-users who are marked as diabetics using any suitable scheme to allocate clinician time and/or organization resources to end-users including but not limited to allocation based on putative new deterioration in gait parameters identified (tentatively) by gait analysis of diabetic end-users' phones' IMUs' data, typically to be explored in clinic thus necessitating allocation of clinician time and/or organization resources such as suitable in-clinic testing.

Operation 1050: Use gait analysis of diabetic end-users' phones' IMUs' data to feed data to classifier/s trained in operation 1010 to screen known diabetics for new appearances of diabetic complication/s known in the literature e.g. neuropathy, retinopathy, nephropathy, cardiovascular disease; flag users accordingly e.g. “retinopathy suspected”.

Operation 1060: Conduct tests (in-clinic and/or using the phone to prompt user to cooperate with remotely administered tests) such as gait assessment and timed-up-and-go to confirm or rule out the suspected complication; mark end-users accordingly e.g. “neuropathy confirmed/ruled out”

Operation 1070: Monitor end-users who are marked as suffering from a given diabetic complication (including post-amputation end-users who suffer from the after-effects of amputation which is considered herein a diabetic complication) using any suitable scheme to allocate clinician time and/or organization resources among the end-users having this complication, including but not limited to allocation based on new deterioration in known after-effects of the known complication—e.g. as identified by gait analysis of diabetic end-users' phones' IMUs' data.

One example of a possible after-effect of amputation (or chain of after-effects) is immobility which may lead to blood clots and possible PE. Amputee immobility which may occur before full recovery or due to failure to achieve full recovery, increases risk for blood clots. once blood pools in the veins, e.g. due to immobility, a resulting clot may travel to the lungs which may lead to pulmonary embolism (PE) especially if the recovery period is prolonged. It is appreciated that one possible measure of an amputee's mobility is the proportion of time in which the amputee's phone's imu data is classified as “gait” which is indicative of mobility as opposed to other time intervals which are indicative of immobility. Users with a high measure of immobility (low proportion of “gait”) may be prioritized by the system for followup designed to promptly identify any signs of PE. Alternatively or in addition, a classifier may be trained to predict pulmonary embolism in amputees, using training data which includes gait analysis parameters and/or imu data from cellphones of amputees known to have been subsequently diagnosed with PE as well as gait analysis parameters and/or imu data from cellphones of amputees not subsequently diagnosed with PE.

Operation 1080: Screen known (advanced) diabetic end-users using gait analysis of diabetic end-users' phones' IMUs' data to feed data to classifiers which identify (advanced) diabetic end-users which may benefit from transiting to palliative care; schedule end-users for consultation regarding transiting to palliative care.

It is appreciated that all or any subset of operations 1040, 1050, 1070,1080 may use standard outcome estimates generated from gait analysis e.g. as described in FIG. 2, e.g. to generate quantitative objective data that may provide a basis for evidence-based decision making, and/or because these estimates may include information about increased motor difficulty which has predictive value but which (absent reliance on the standard outcome estimates as generated herein) may never reach the attention of clinicians due to frequent patient failure to report these increased difficulties and/or due to insufficient clinician testing e.g. due to insufficient personnel/equipment resources.

For example, in operation 1040, standard outcome estimates generated from gait analysis e.g. as described in FIG. 2 may be used as a basis for clinician allocation e.g. by prioritizing end-users whose standard outcomes have deteriorated to a greater extent since their last clinic visit, over end-users whose standard outcomes have deteriorated to a lesser extent since their last clinic visit, using any suitable logical and/or computational formula to combine deteriorations for each individual standard outcome estimated. Deterioration of a given estimate standard outcome since the last clinic visit is possible to compute, because the estimates are computational thus the system may compute current standard outcomes (time-stamp=today) and subtract same from respective standard outcomes on (known) date/s of each user's last clinic visit.

In Operation 1050, standard outcome estimates generated from gait analysis e.g. as described in FIG. 2 may be used as a basis by which to screen for new appearances of diabetic complication/s e.g. if certain standard outcome scores are known to be typical of certain diabetic complications.

In Operation 1070, standard outcome estimates generated from gait analysis e.g. as described in FIG. 2 may be used as a basis by which to screen for known after-effects of a complication that a user is known to suffer from e.g. if certain standard outcome scores are known to be typically of certain after-effects.

In Operation 1080, standard outcome estimates generated from gait analysis e.g. as described in FIG. 2 may be used as a basis for identifying (advanced) diabetic end-users who may benefit from transiting to palliative care.

It is appreciated that clinical guidelines, standards of care and best practices, and/or insurance coverage, may be defined in terms of standard outcomes. For example, a geriatric society may recommend that individuals with a TUG score above 20 seconds, should be referred to a physical therapist for strength training of lower body muscles and balance exercises (e.g., standing on one leg, Tai Chi) whereas individuals with a TUG score below 20 seconds are not so referred. Or, an onlineguide, |CDC STEADI: Best Practices for Developing an Inpatient Program to Prevent Older Adult Falls after Discharge” instructs to “refer to PT if patient fails test (e.g., TUG>12 seconds, inability to complete number of chair stands for sex and age in 30-Second Chair Stand test, inability to hold at least a 10-second tandem stance in 4-Stage Balance Test”.

These standard outcomes conventionally need to be collected for each patient by enlisting the patient and/or her or his clinician to expend time and effort in personally/manually generating each standard outcome score (e.g. patient filling in questionnaire, clinician administering test to patient, etc.). According to embodiments herein, these standard outcomes are estimated automatically e.g. as described herein with reference to FIG. 2, rather than being personally/manually generated.

Certain embodiments seek to provide a health management system comprising:
a. A personnel allocation processor for allocating personnel to conduct home or clinic sessions with diabetes patients; and/or
b. A gait analysis processor receiving IMU data from cellphones worn by the diabetes patients and providing the personnel allocation processor with indications of a subset of the diabetes patients whose IMU e.g. accelerometer data indicates new deterioration.

Typically, allocation of personnel by the personnel allocation processor at least partly favors diabetes patients falling within the subset over diabetes patients who do not fall within the subset.

The term “at least partly” is intended to include systems characterized in that, all other things being equal, the system favors diabetes patients falling within the subset over diabetes patients who do not fall within the subset, in the sense that patients within the subset are likely to receive earlier and/or more staff attention than patients not within the subset. However, it is not necessarily the case, given two specific diabetes patients, who are equal other than their cellphone data, that the patient falling within the subset will be favored over the patient who does not fall within the subset. This favoring may occur for some such pairs of patients, and not for others.

The term “at least partly” is also intended to include systems which allocate staff to patients, partly based on whether or not given patient IMU e.g. accelerometer data indicates new deterioration, and partly based on other considerations.

A particular advantage of the embodiments herein is that there is typically a need to follow up diabetes patients essentially for their entire lives, because their glucose levels may have gotten out of balance, and or because they may be developing symptoms, which, if untreated, may result in complications, which may lead to amputation. The known way of accomplishing this is to follow up, in an equal manner, all diabetes patients known to the organization. Thus, the personnel allocated by the system may see George on a specific day, and may next see George only after available staff has seen all other patients, and George's turn has come round again, e.g. in round-robin fashion, or an approximation thereof. Also known is the method of relying on human intuition and discretion exercised by the domain experts, e.g. human administrators of the available staff, to prioritize some patients over others, including, say, that, based on a chance phone call with George, the human administrator may believe correctly or incorrectly that an appointment for George is more urgent than other diabetes patients known to the organization who are also waiting for appointments.

In contrast, according to embodiments herein, the organization may allocate its personnel to follow up diabetes patients known to the organization, by prioritizing at least some patients over at least some others, on the basis that some patients' cellphone data, which is available to the system, e.g. via a cell app that users have enlisted to, is indicating motor deterioration relative to what is considered that patient's baseline, and other patients' cellphone data is not indicating any such motor deterioration relative to their respective baseline. Alternatively, the organization may combine some or all of the above three ways, either automatically or intuitively, by means of human judgment on how to combine them.

It is appreciated that the following situations, if undetected and untreated, are likely to result in limb amputation in diabetes patients. Yet, the cellphone data reaching the system may be used to screen for people who seem to be developing one of these situations.

In diabetes patients, undetected and untreated complications may lead to situations that may result in limb amputation. The most common factors that contribute to amputation are diabetic neuropathy and poor circulation.

Diabetic foot ulcers, for example, may increase the likelihood of amputation if left undetected and untreated. The system may train a classifier to detect diabetic foot ulcers e.g. using training data which includes imu data (or gait parameters derived therefrom) for some users known to be suffering from such foot ulcers and for other users (eg diabetics) known not to be suffering from such foot ulcers.

Thus according to certain embodiments, a monitoring method for diabetics may be provided which includes, offline, training a classifier using training data including historical imu data generated by users later diagnosed in-clinic as suffering from a foot ulcer (or charcot foot), and also including historical imu data generated by users later seen in-clinic without being diagnosed as suffering from a foot ulcer (or charcot foot).

If the system succeeds in identifying a suspicion of such foot ulcers, which may become infected, the system typically initiates a sequence of operations to enable the end-user to be contacted and/or to be promoted to upload an image of her or his foot and/or to be seen face-to-face and/or to be treated to prevent infection of the ulcer, since, if the infection is not treated, it may spread and cause severe tissue damage, and may even reach a level where amputation may be necessary to prevent the infection from further spreading to other parts of the body.

It is appreciated that gangrene occurs when a body part, usually a toe, foot, or part of the leg, loses blood supply and the tissue begins to die. This may be a result of untreated infections, or nerve damage inter alia. Barring prompt proper treatment, gangrene may spread to surrounding tissues, making amputation necessary. Thus, according to certain embodiments, an image of a foot which a non-diabetic user, who has been flagged as possibly suffering from diabetic foot ulcers, may responsively have been prompted to capture, may be automatically sent to image processing, which may be configured to identify foot discoloration (blue, black, or dark red skin).

Any suitable method may be employed for identifying such foot ulcers. For example, a classifier may be trained to identify changes or deterioration, e.g. an increase in asymmetry, or decrease in the case of gait speed in spatiotemporal parameters which tend to occur concurrently with the appearance of foot ulcers, such as but not limited to all or any subset of the following parameters: double support stance asymmetry, step length asymmetry, cadence, cadence variability, and gait speed.

It is appreciated that automation of the detection of diabetic foot ulcers is particularly important because it sometimes occurs in diabetics whose nerves are damaged by the disease, aka diabetic neuropathy, particularly in the feet and legs, typically adversely affecting the entire sensitivity to pain and the feedback loop of sensing, which is also an important component in the gait control mechanism.

Thus, gait pattern changes in diabetics, which are detectable via spatiotemporal parameters, may, according to certain embodiments, be detected as two separate classes, given that the gait pattern may change differently, depending on whether the change occurs because of pain, or because of the foot ulcer itself. According to certain embodiments, the system herein performs gait analysis which may distinguish between the two cases (pain or loss of pain/sensing). The system may collect data from these two groups of patients and train a model that inputs their gait pattern, or the mobility longitudinal data, and outputs a classification which may differentiate foot ulcers with pain, from foot ulcers with loss of pain, and, in a third category, those diabetics who are fortunate enough not to suffer from foot ulcers at all at the time.

Another class which may be differentiated by the classifier is that of end-users who are experiencing Peripheral Artery Disease (PAD) which also leads to pain in the legs, particularly during physical activity, numbness, or a feeling of coldness in the legs or feet. Thus, the system may define, as classes, Peripheral Artery Disease (PAD) associated with pain in the legs, particularly during physical activity and/or Peripheral Artery Disease (PAD) associated with numbness which is also expected to manifest itself in a different gait pattern change relative to other classes described above.

Periods of physical activity vs. periods with no such activity may be identified by identifying time intervals in which gait is occurring as opposed to time intervals in which gait is not occurring e.g. as described in co-owned U.S. Ser. No. 18/975,890 (hereby incorporated herein by reference in its entirety) which is entitled “Advanced Pedestrian Navigation Based on Inertial Gait Analysis and GPS Data” and filed 10 Dec. 2024 which describes how to identify “gait” type time-intervals and/or in co-owned US 2022/0111257 (hereby incorporated herein by reference in its entirety) which is entitled “Efficient System Configured To Facilitate Physical Rehabilitation”.

It is appreciated that alternatively, or in addition, the system herein may include machine learning capability, and may be trained, for example, to perform gait analysis to identify gait changes, and accordingly to predict results of direct testing such as the ankle-brachial index (ABI) test that measures blood flow in the legs to identify low ABI results, which indicate poor circulation, or ultrasound or angiography imaging tests, which evaluate the extent of arterial blockages.

According to certain embodiments, the system is trained to identify users suffering from pain in the legs (e.g. during physical activity specifically) and/or is trained to identify users suffering from numbness. A classifier may, for example, be trained on tagged data including end-users complaining of leg pain, particularly during physical activity and/or end-users complaining of numbness; the system may, for example, look back at earlier IMU data, e.g. in the days before such leg pain was reported, perhaps even determining how many days to look back, by asking the end-users when the leg pain started. Once a classifier is trained on this data, the classifier is then able to classify end-users into two classes, namely a first class of end-users who are suffering from leg pain and/or numbness, as opposed to a second class of end-users who are not suffering from such leg pain and/or numbness. The system may also comprise a classifier trained to differentiate between end-users who have swelling, warmth, or pus around a wound, from those who do not. For example, given end-users who report swelling (or warmth, or pus around a wound) to their healthcare provider (or as part of using the app which implements any embodiment herein). These reports may be used as dependent variables, and earlier measured gait parameters may be used as independent variables.

Another class which may be differentiated by the classifier is that of end-users who are suffering from Charcot Foot (Neuropathic Arthropathy) which occurs when nerve damage leads to weakening of the bones in the feet. The feet may become deformed, and the joints may break down. This classification may be performed at suitable intervals, particularly on end-users who have already been identified, whether by a human medical professional or by the system herein, as suffering or likely to be suffering from diabetic neuropathy. It is appreciated that, if left untreated, Charcot foot may result in a significant risk of infection and an increased probability of subsequent amputation.

It is appreciated that if an end-user is identified as belonging to this class at a given threshold level of confidence, the system may again request an image of the foot so as to corroborate (or not) the system's classification by using image processing to identify changes in foot shape or alignment relative to previous images in the system of this same user's foot, if and as available.

For example, assume that the system has generated a general lower body pain score based on any mobility data available (eg as described herein with reference to FIG. 2). This score was trained to estimate the standard clinical outcome for measuring lower body pain in diabetic patients including identification and use of trends.

This general lower body pain score may be used for all or any subset of the following applications:
1. Disease management—Should a specific patient get an in-person appointment to check for foot ulcers or other diabetes complications that may result in amputation?
2. Should any change in treatment or drug dosage be made?
3. May be used in a clinical trial of a given drug to compare different groups of patients undergoing different treatment (control group, placebo, the examined intervention)
3. pharma post-marketing surveillance to reliably demonstrate say that patients using the drug retain good mobility and independence for longer.

It is appreciated that the system herein may interface directly with suitable doctors' office software for managing appointments, such as, say, Zocdoc which provides prioritization, which may be determined by the type of appointment (emergency, routine, etc.), such that cases flagged by the system herein may be automatically defined as emergencies, rather than as routine, which then allows such urgent care options to be prioritized for immediate appointments.

Similarly, Practice Fusion is an EHR (Electronic Health Record) platform that includes integrated appointment scheduling. It allows for both patient self-scheduling and staff scheduling, and it integrates reminders.

The platform has priority logic via which staff may assign priority to appointments based on clinical urgency (e.g., a patient requiring immediate consultation), and, responsively, the software may prioritize appointments with doctors, based on the severity of the situation. According to embodiments of the invention, the priority assignment may be performed by the system based directly on gait data arriving from patient cellphones, and there may be no staff involvement, or staff approval may be required, or staff may override any data received.

Pre-trained machine learning models may be used by the system to estimate the plausibility (e.g. probability that a certain binary indication such as lower body pain (yes/no) is present) of any of the following Diabetic indications or standard outcomes. This plausibility data may be used for screening for diabetes in the general population and/or for diagnosing or screening for diabetes complications e.g. in the course of tracking and monitoring of known diabetics Indications may for example include lower body pain indications, instability and fall risk indications, cognitive impairment indications.

Any suitable training data may be used during training e.g. when seeking to train a model to estimate Lower body pain indication all or any subset of the following may be employed: pain rating, EQ-5D, and LEFS scores collected by the apparatus as labels. A regression model (say) may be trained to output all or any subset of these scores. Previous reports of pain, asymmetries and variabilities in gait measures (such as stance asymmetry), and dual task cost measures or any subset thereof may be used to indicate pain.

When training a model to estimate Instability and fall risk indication all or any subset of the following may be used: previously reported falls, EQ-5D, Falls Efficacy Scale, and LEFS scores collected by the apparatus e.g. clinician-facing dashboard as labels. The regression (say) model may be trained to output all or any subset of these scores. Previous reports of falls, asymmetries and variabilities in gait measures (such as stance asymmetry), and dual task cost measures, or any subset thereof, may be used to indicate instability and risk of falls.

It is appreciated that any suitable output indication generating functionality may be employed to generate an output indication of any result or prediction or parameter described herein, which may be yielded by any embodiment of the system herein; the output indication may be provided to any suitable entity in any suitable manner (e.g. automatic SMS to HMO and/or a report document which may be consumed via web dashboard or via API with EMR software platform such as Epic Systems or Cerner, or via email to an email address, stored in the system, of the entity).

It is appreciated that the method of FIG. 1 may be modified to include all or any subset of the following functionalities:

It is appreciated that episodes of hypoglycemia (low blood sugar) or hyperglycemia (high blood sugar may occur inter alia due to changes in physical activity. According to certain embodiments, the system may predict hypoglycemia or hyperglycemia e.g. based on gait speed changes and/or balance changes, e.g. lower estimated scores for known balance tests, such as a Romberg test.

It is appreciated that exercise is beneficial for diabetes patients. However, fatigue or joint pain may result in insufficient physical activity. Certain embodiments of the system herein detect factors which tend to result in insufficient physical activity, and trigger alerts to professional system users accordingly. For example, the system may use standard mobility measures e.g. standard diabetic outcome measures, such as the self-reported standard questions in the first part of Michigan Neuropathy Screening Instrument. It is appreciated that embodiments herein which aid in identifying potential blood sugar level disruptions, e.g. by identifying insufficient physical activity, are particularly advantageous for demented patients who have difficulties in identifying these problematic states, e.g. due to difficulty in recognizing and/or communicating symptoms such as shakiness or confusion.

Hypoglycemia (low blood sugar) and hyperglycemia (high blood sugar) may be detected, according to embodiments herein, using motor symptoms that gait analysis may identify, for example:

### Hypoglycemia:

1. Tremors/Shakiness: especially in the hands; the system may prompt the patient to hold the phone in her or his hand, and the system may then record motion; co-owned U.S. Ser. No. 18/975,890 entitled “Advanced Pedestrian Navigation Based on Inertial Gait Analysis and GPS Data” and filed 10 Dec. 2024 (hereby incorporated herein by reference in its entirety) describes how to identify “shakes” type time-intervals as opposed to, say, gait intervals, where the former may indicate an end-user who is experiencing shaky hands, and the latter may indicate an end-user who is not experiencing this problem.
1. Coordination problems: e.g. clumsiness, or difficulty walking (ataxia), which the system may identify as gait variability and symmetry.
2. Weakness or fatigue which the system may identify as a low score on a 6-minute walk test, relative to a patient's own norms or relative to population norms.

### Hyperglycemia:

1. Weakness or fatigue e.g. as described above
2. Slow movements or slow reactions which the system may identify as low gait speed relative to a patient's own norms, or relative to population norms.

Typically, Hypoglycemia may be predicted for more immediate and acute motor symptoms, whereas Hyperglycemia may be predicted for more gradual motor symptoms.

It is appreciated that prompt detection of extreme blood sugar disruptions is important, given that these, if extreme, are emergency situations requiring rapid intervention. Even the best of caregivers or patients may not be aware of or may misinterpret warning signals, whereas embodiments herein are reliable and consistent in flagging symptoms.

### EXAMPLE

Common signs of DKA that may precede motor or neurological symptoms include:
Excessive thirst (polydipsia)
Frequent urination (polyuria)
Nausea and vomiting
Abdominal pain
Fruity-smelling breath
Rapid, deep breathing (Kussmaul breathing)
Confusion or altered mental state

If any of these signs are observed in a diabetic patient, urgent medical care typically must be sought to prevent severe complications, including motor and neurological dysfunction. Unfortunately, some patients may not recognize these classic symptoms, and if their caregivers are remote, the system may fail to flag the DKA condition and fail to provide the needed urgent medical care. It is appreciated however, that Diabetic Ketoacidosis (DKA) may also involve motor dysfunction, thus yielding motor signs which may be detected according to embodiments herein, and used as remote warning signs of DKA, e.g.:
1. Muscle weakness, which may affect the ability to move and control limbs effectively as evidenced by slow gait speed, relative to any suitable threshold stored in the system.
2. Tremors or shaking which may be detected, e.g. as described herein.

A classifier may be trained to identify passive walks which suggest DKA e.g. by using training data which includes some passive walks taken by known DKA patients, and other passive walks taken by end-users known not to be suffering from DKA.

Diabetic neuropathy is a common diabetic complication. Regular foot checks may not occur e.g. because of difficulties in coordinating between the many clinicians required to properly manage diabetic care, particularly in underserved areas and/or populations. Also, neuropathy may be diagnosed via patient complaints (such as pain, tingling, or weakness) which prompt clinicians to order clinical tests. However, demented or elderly or depressed or otherwise compromised persons, for example, may never communicate such complaints, and may omit to report increased difficulty in walking.

The system may learn gait patterns typical of patients with this kind of damage by using training data, to train a classifier e.g., which includes examples of gait derived from patients known to suffer from this complication, and examples of gait derived from patients known not to suffer from this complication. Tests for neuropathy may be automatically ordered by the system for patients whose gait analysis reveals gait patterns typical of neuropathy, and typically not for patients whose gait analysis does not reveal such patterns.

Specifically, neuropathy, or nerve damage, may cause the following; classifier/s may be trained to detect gait patterns typical of sufferers from all or any subset of the following symptoms:
1. Allodynia
2. Muscle cramps or weakness, or a dull, constant ache
   1. It is appreciated that to train such a classifier, training data may include gait measurements from, say, 100 patients known to be suffering from diabetic neuropathy complication/s affecting gait, e.g. all or any subset of the above symptoms, and from, say, 100 healthy people.
   A classifier may then be trained to distinguish between gait patterns from these two groups.

A single classifier may be trained to identify difficulty walking, which is typical of any early sign of diabetic complications, such as all or any subset of:
Peripheral Neuropathy: Pain, numbness, weakness, or balance issues in the legs and feet.
Peripheral Artery Disease (PAD): Pain, cramping, or weakness in the legs, especially during activity.
Charcot Foot: Foot deformities and instability.
Musculoskeletal Issues (e.g., Osteoarthritis).
Retinopathy: Vision issues that make walking difficult.
Autonomic Neuropathy: Dizziness or fainting due to low blood pressure.
Foot ulcers or infections.
Or, a separate classifier may be trained to identify a specific individual symptom, e.g. from among the above.

Given the increased likelihood of hospitalizations due to complications like infections, cardiovascular events, or severe blood sugar imbalances, thus decreased likelihood of hospitalizations to the extent that early detection of complications like infections, cardiovascular events, or severe blood sugar imbalances may be achieved, embodiments herein are seen to be particularly advantageous.

It is appreciated that it is desired to detect undesirable interactions between diabetic medications and other drugs, or even between different diabetic medications, but these may also cause motor symptoms such as but not limited to muscle weakness, tremors, coordination problems, or even more severe manifestations such as difficulty walking. For example, diuretics (often prescribed for hypertension or fluid retention), may lead to electrolyte imbalances, which may disrupt nerve and muscle function, leading to:
Muscle cramps
Weakness
Tremors or twitching
Difficulty with movement and coordination
All or any subset of of the above may be detected by the system herein, according to any embodiment described herein e.g. training suitable classifier/s.

An advantage of certain embodiments is that, since diabetes requires constant self-monitoring and decision-making, patients and/or their caregivers may, over time, experience “diabetes burnout,” i.e. being overwhelmed by the daily demands of managing their condition; embodiments herein are greatly advantageous in reducing this burden on the patient by providing automatic monitoring which may at least partly replace conventional self-monitoring by the patient herself/himself. This is especially true of patients, aka end-users, who suffer the double burden of, say, diabetes and dementia. The treatment burden may itself cause or worsen depression and anxiety which are common in demented persons. While diabetes management has already been augmented by dedicated technological tools such as CGMs (continuous glucose monitors), these devices are costly for some, and are thus inferior to embodiments herein, which rely only on the use of a cellphone. Example: 150 minutes per week of moderate-intensity exercise aids in stabilizing glucose levels. The system herein may passively monitor for moderate intensity exercise, and flag patients who are not undergoing 150 minutes per week of moderate-intensity exercise.

Another functionality that may be provided is functionality to facilitate prevention of unnecessary amputations in diabetes patients. For example, if diabetics known to the system are labelled each time a foot infection or foot ulcer culminates in an amputation of the same foot, this may enable a classifier to be trained, using this system information regarding diabetics who have had foot infection followed by amputation, as opposed to a class of diabetics who have not suffered from foot infection and/or who have not had to undergo amputation. Foot infections, common in diabetics e.g. due to poor circulation and neuropathy, may yield motor symptoms e.g. pain when walking, pain when standing, foot swelling which hampers movement, and numbness leading to problems with walking or even gangrene, which may result in muscle weakness, hence difficulty using the affected foot or leg. Detection of rise or dip in blood glucose levels also prevents amputations, given these improper glucose levels, which may yield complications that may result in amputation. Better diabetes management, e.g. as described herein, also prevents amputations. It is appreciated that a classifiers may be trained to detect diabetic foot ulcers, thereby reducing unnecessary amputations, e.g. by screening for diabetic neuropathy gait patterns or changes in gait mobility measures, and alerting for provision of clinician attention for the patient in question, raising diabetic foot ulcers as a possible cause.

Another functionality that may be provided is ongoing monitoring for circulatory problems such as Peripheral Artery Disease (PAD), whose motor symptoms may be used to facilitate early detection which may help in the early detection of these issues, especially in people with conditions like diabetes. Symptoms that may be detected by suitable gait analysis which may include training a suitable classifier, e.g. as described elsewhere herein, may include identification of all or any subset of the following motor symptoms which may be indicative of circulatory problems, e.g. Peripheral Artery Disease (PAD):
1. Claudication (Leg Pain with Activity) e.g. pain or cramping usually in the calves, thighs, or buttocks, that occurs during physical activity (walking, climbing stairs) and improves with rest. Since the system herein is able to detect periods of walking, stair-climbing, and rest, the system may identify this symptom which is considered a major motor symptom that signals reduced circulation. Other symptoms include:
   Muscle weakness or fatigue which may render walking or standing for long periods more difficult
   Legs giving out or becoming weak when trying to walk, climb stairs
   Difficulty maintaining balance
   Lack of coordination.
2. Numbness or tingling which may lead to a higher risk of falling or tripping, including:
   Reduced ability to perform walking, running, or climbing stairs
   Weakness in the legs when standing or walking.

According to certain embodiments, if the above motor symptoms are identified, the end-user may be flagged for clinician attention, and, inter alia, may be prompted to capture images of her or his legs (say) to enable image processing to identify signs of PAD, such as:
Shiny skin, or hair loss on the legs
Skin color changes (paleness or a bluish hue).

It is appreciated that early detection, e.g. as described herein, is advantageous in allowing early introduction of all or any subset of lifestyle changes, medications, and surgical procedures, whereas failure to detect peripheral artery disease (PAD) or other cardiovascular issues, may severely affect mobility, and even survival, due to increased risk of heart attack and stroke and/or due to poor circulation, especially in the legs and feet, causing gangrene, particularly in the feet, which may result in severe infections and may necessitate amputation.

It is appreciated that the system typically tracks a single patient's gait data over plural stages of diabetes which is more efficient than having one computerized system (e.g. in a first health-providing organization) which records patient data (including gait data e.g.) for one stage of diabetes or for one purpose, e.g. for a first specific service, this health organization happens to provide to diabetes patients-typically inter alia, and another computerized system (e.g. in a second health providing organization) which records patient data (e.g. gait data) for another stage of diabetes or another purpose e.g. for a second specific service this second health organization happens to provide to diabetes patients-again typically inter alia. Having a single system track each diabetes patient's gait data through various stages and over various purposes enables the system to derive how patient-specific characteristics e.g. genetics and/or lifestyle and/or her/his specific pattern of comorbidities, influence disease progression, as evidenced by gait data. A lesser level of granularity may result if separate computer systems were to be used. Alternatively or in addition, earlier stages of the illness may affect later stages, and the system herein may then be able to machine-learn these relationships between early stage gait data and later stage gait data, whereas separate systems for separate cohorts may fail to machine-learn these relationships and thus be less able, or unable, to generate predictions that the system herein may be trained to generate, over time.

This results in a more efficient system relative to separate systems, e.g. because given a certain number of end-users, more relationships may be learned and/or more meaningful insights may be gathered, relative to separate systems each handling the same number of end-users.

Any suitable machine-learning may be employed to teach the system herein to predict later gait data and/or outcomes of later-stage treatments, from earlier data e.g. earlier-stage gait data collected by the system and/or standard outcomes that the system learned from earlier-stage gait data. Any suitable training process may be employed for training a model to understand such patterns over time, where earlier observations (features) are used to predict later outcomes (target variables). For example:

Data preparation aka pre-processing may include structuring longitudinal data such that each row represents a time point for a given individual e.g. one row per patient per time point (e.g., patient 1 at time 1, patient 1 at time 2, . . . , patient 2 at time 1, . . . patient n at time 2, . . . etc.). For each time point, the model may receive input features (e.g., characteristics and/or measurements and/or gait parameters characterizing a given end-user who bears a cellphone which has a built-in IMU) and may receive a target (e.g. outcome to be predicted, such as some concurrent or later measure of diabetes progression, e.g. any standard outcome used for this purpose) for the same end-user which is associated in memory with the input features, hence may be the label for those input features or independent variables.

The model may, for example, include Recurrent Neural Networks (RNNs)/Long Short-Term Memory (LSTM) configured to handle sequential data in a time-based prediction context by retaining data from earlier time points to predict future outcomes and/or by learning temporal dependencies in the data. Random Forests or Gradient Boosting Machines (GBMs) may be used, e.g. if data is transformed into a feature set representing the diabetes patient's history up until a specific time-point, and then the model is trained, based on these features, to predict target/s at later time-points. Features that capture temporal aspects may be included, e.g. moving averages and/or differences between time points. Linear regression/time-series models, e.g. ARIMA, may be used, typically with temporal features such as lagged variables (previous time-points) used to predict future values, such as future standard outcomes for diabetes, at later time-points. Survival Models, e.g. Cox Proportional Hazards, or other survival analysis techniques, may be employed to predict a time-to-event outcome (e.g., time to disease onset and/or progression) which may be useful.

Lag features may be created, typically representing values from previous time points to enable the model to learn temporal dependence. Rolling window features may be used to summarize data over a time window (e.g., average of a given gait parameter over the past three days, or change in this gait parameters value over the last P IMU-sampling points). To determine how rapidly a feature is changing over time, the system may compute differences (e.g. in a given gait-parameter) between consecutive time points or the slope of the feature over time.

Typically, the model is trained on earlier time points and tested on later time points to avoid “look-ahead bias”. In time-series problems, time-series cross-validation may be used.

The model's performance may be evaluated using any suitable metric/s such as but not limited to accuracy, RMSE, AUC, or survival analysis-specific metrics, using a test set with future time-points that is separate from the data used to train the model. If interpretability is important, e.g. due to a need for justification documentation which justifies treatment decision, models with easily interpretable features such as decision trees or linear models may be employed.

According to certain embodiments, standard outcomes at a given time T, such as but not limited to PROs (patient-reported outcomes), clinician test results, and even lab results, may be predicted by the system, typically by combining plural measurements e.g. gait parameters or IMU data over time e.g. including measurements e.g. gait parameters collected at times which precede time T. The plural measurements may be generated by gait analysis which may include all or any subset of:
1. Spatiotemporal analysis: evaluation of gait properties such as gait cycle time, cadence, gait speed, stride length, right and left step length, base width, right and left single support and stance time and percentage, double support time and percentage e.g. as described in co-owned US 2020/0289027 A1 (hereby incorporated herein by reference in its entirety)
2. Kinematics: including any of the lower body joint angles over the gait cycle
   e.g. as described in co-owned Ser. No. 17/666,180 (hereby incorporated herein by reference in its entirety)
3. Kinetic: evaluation of gait properties related to torques and forces such as ground forces and center of feet pressure over the gait cycle, and the analysis of its sway over the gait cycle
4. Variability: the deviation of any of the gait properties above between different strides over the same walk. May be processed in two forms, e.g. aggregated as the standard deviation of each value and/or as a sequence of the differences in values over time.

The plural measurements may be combined e.g. by performing all or any subset of the operations shown in FIG. 2, in any suitable order. Thus FIG. 2 includes a diabetic indication assessment process which is configured to estimate diabetic indications, considering all longitudinal data and patient information, e.g. as described below. The operations of FIG. 2 may include all or any subset of the illustrated operations, suitably ordered e.g. as shown. Specifically:

810. Gather all patient data within a suitable time-window e.g. all historical data in the system or only data for the last 1-2 year/s, typically including all information provided on the dashboard or using the apparatus e.g. all or any subset of the following: demographic information, clinical conditions, treatment log, active and passive gait analysis, standard mobility test analysis, patient-reported outcomes and questionnaires, clinical events, injuries, surgeries, and medication. This is typically performed on the cloud, whereas all or any subset of operations 820-870 may be performed locally. It is appreciated that for prediction of standard outcomes, data used may include data derived from the patient's phone's imu and/or Mobility measures which may be obtained in-clinic (e.g. obtained from TUG or other mobility tests which may for example characterize the patient's standing, sitting, turning etc.

820. Filter outliers and disrupted data from the data gathered in operation 810-sometimes, there may be some discrepancies in estimated mobility measures e.g. in gait analysis parameters and mobility test measures. This is even more likely in passive gait measurements, e.g. since environmental effects may be unknown. To reduce such noisy data, it is recommended to filter out (or at least indicate with lower certainty) strange data points aka outliers. This task is possible when the patient's timeline is available, which enables the comparison of data points and the patient's reference. For example, a gait speed higher or lower by three standard deviations from the patient's weekly average gait speed may be considered an outlier. When many data points are available, as in passive gait monitoring, a daily or weekly (or any other timespan) aggregation, taking the median or the average, may be a solution to reduce outliers' noise.

830. Interpolate trends in the filtered data generated in operation 820. Mobility measures (such as gait speed, stance asymmetry, ranges of motion, and PROs (patient-reported outcomes) typically have a characteristic behavior which is continuous over time, e.g. changes in these measures are not usually dramatic, and thus the system may interpolate between data points. For example, if an end-user has recovered from a condition, and his/her gait speed the previous week was 0.7, and this week is 0.9, then somewhere in between the gait speed may be assumed to have been 0.8, unless a dramatic event has occurred (such as an injury). It is thus reasonable to interpolate some mobility measures between data points, as long as no medical incident has occurred, and produce trends for those measures. Linear interpolation between data points, e.g. of the data points collected in operation 810 (for each measure), and then filtered in operation 820, may be carried out, and then averaging the resulting interpolated data over a sliding window, to reduce measurement fluctuations afterward. The more frequent the measurements, the more representative the trend line. Gait active measurements may be taken between a few times a week and once every two weeks, depending on the natural progression of the patient's condition. The sliding window length may be determined accordingly to match the measurement frequency required in the program by the patient. For example, if a patient produces a few walks a day, the sliding window may be a day or two-day window, so that it covers several measurements. If a patient fills out one questionnaire a week, the sliding window's length may be 2 to 4 weeks.

840. Modify norms and/or increase norm granularity/specificity: norms are aggregations of patients' data and timelines over a specific population to compare to the patient's measures, indicating whether the patient is above or below the norms, for example gait parameter norms by demographic data, such as normative values of gait speed by age and gender. Another example is the recovery pace in different parameters or outcome measures post-surgery, for instance benchmarks of 6-minute walk test distance by week for different surgeries and populations. Norms and benchmarks may be hardcoded, supported by literature, or computed by observations. Hence, when new data is collected and the method of FIG. 2 is executed, there is an opportunity to regenerate norms. This process may alternatively run after a large set of new timelines and patient data is collected. Norms may become as granular and specific as available, e.g., gait norms for Parkinson's patients by gender, age, height, and weight.

850. generate a Feature vector representing the patient status at a specific date e.g. patient 345 in day 2025 Feb. 3. This may include a set of selected variables, e.g. the outputs of operations 810-840 may yield a vector representing a given patient's data at a specific timestamp (or date), in which each index allocates a specific variable or has a value which equals a corresponding parameter interpolated value. These variables are derived from any or all of the outcomes and measures of the gait analysis, questionnaires, standard mobility tests, demographics, and norms. One alternative is to define the variable value or to define a variable's value at a specific time-stamp on which the variable was not measured, to assign the corresponding measure's/variable's interpolated value at the specified timestamp. Another alternative is to set the value to be the actual value as last measured; typically, the system may add a representation (e.g. number of days including fraction) of the time delta since this variable was last measured. The measurement's time may be embedded e.g. as described in arxiv.org/pdf/1907.05321. which describes providing a model-agnostic vector representation for time.

860. Estimate a standard outcome e.g. by using pre-trained machine learning models. Any suitable method may be employed to train the models, e.g. using output of operation 850 and/or mobility measures and/or patient data 810, filtered e.g. as in operation 820, interpolated e.g. as in operation 830, compared to norms e.g. as in operation 840, represented at a specific date e.g. as described herein with reference to operation 850, and used in the ML models. This may be labelled to indicate known levels of standard outcome (e.g. IMU data of persons known to have normal sit-to-stand test results, plus IMU data of persons known to have abnormal sit-to-stand test results, may be used as training data for a classifier being trained to estimate sit-to-stand results) e.g. as described elsewhere herein. The models may include regression models, such as but not limited to SVM, linear regression, neural network regression models, decision tree-based models, random forest, and XGBoost. Operation 860 typically uses pre-trained machine learning models to estimate the plausibility of diabetic indications, such as but not limited to any diabetic indication described herein, including any of the following indications: lower body pain indication, instability and fall risk indication, cognitive impairment indication, any gait parameter which may be indicative of diabetes, e.g. as described herein.

According to certain embodiments, the feature vector generated in operation 850 is used as the sampling space or input samples to train a model which, once trained, predicts various labels Such as pain rating, EQ-5D, and LEFS scores.

Training may, for example, include using lower body pain indication e.g. using pain rating, EQ-5D, and LEFS scores collected by the apparatus as labels. The regression model may be trained to output any subset of these scores. More generally, any suitable method may be employed to train models typically one model for each indication or standard outcome to be predicted.

Previous reports of pain, asymmetries and variabilities in gait measures (such as stance asymmetry), and dual task cost measures, are expected or likely to indicate or predict subsequent pain (e.g. a day later). More generally, any other parameter/s mentioned in this specification may be used for training a diabetic indication classifier such as, say, a1c levels (aka hemoglobin a1c or hba1c blood test, used to diagnose prediabetes and diabetes).

Alternatively, or in addition, the system may use instability and fall risk indication, e.g. may use previously reported falls, EQ-5D, Falls Efficacy Scale, and LEFS scores collected by the apparatus e.g. via the clinician-facing dashboard as labels. The regression model is trained to output any subset of these scores. Previous reports of falls, asymmetries, and variabilities in gait measures (such as stance asymmetry), and dual task cost measures, are expected to indicate instability and risk of falls.

Alternatively, or in addition, the system may use cognitive decline indication, e.g. may use previously reported A1c results and CASI scores collected by the apparatus e.g. clinician-facing dashboard as labels. The regression model is trained to output any subset of these scores. Deterioration in gait quality (such as a decrease in gait speed or increase in variability) and dual-task cost measures, are expected to indicate instability and risk of falls.

Alternatively, or in addition, the system may use score thresholds, e.g. if any scores in the output subset are in the pathological or non-normal values, the diabetes indication may be included in the assessment output. Otherwise, this indication is considered inapplicable.

865. Alternatively or in addition to performing operations 850 and/or 860, and/or rather than treating the machine learning problem as a regression task from a feature vector into a predicted score, the standard outcome measure prediction task may be treated as time series forecasting that gets observations, typically within the 1-2 year/s that precedes the current time-stamp, each corresponding to a different timed measure or a static data point (gender, condition, etc.). In this case, every observation e.g. each data point comprising a mobility measure from operation 810 may include a time representation (0 may be used when the variable e.g. gender, patient clinical condition has no timestamp), the variable type representation, and the value representation, e.g. as described in arxiv.org/pdf/2109.12218. A model may be trained on this time series of observations and may then be applied to estimate all or any subset of the following indications at a current timestamp: lower body pain indication, instability and fall risk indication, cognitive impairment indication, or any other indication described herein.

870. If an indication score aka predicted standard outcome instead falls within a system-defined pathological range, include the indication score representing the standard outcome measure in the assessment's output. Typically, if an indication score is lower than threshold or falls below the system-defined pathological range, no indication or alert need be provided e.g. as described forthwith.

890. Present the output of operation 870 and/or preceding operations, on the apparatus e.g. clinician-facing dashboard, including all predicted standard outcomes whose values are higher than system-stored thresholds. On a system dashboard, the system may recommend to a clinician end-user or automatically trigger action/s such as but not limited to all or any subset of: call patient, conduct in-person evaluation, change dosage, or treatment.

It is appreciated that the outcomes predicted by the system herein are not limited to the 6MWT (Six-Minute Walk Test) or other (mostly diabetes-specific) outcomes described above. Any outcome used for diabetes screening and/or diagnosis and/or management may be predicted within the scope of the present invention, but more generally, any standard outcome used by any healthcare professional for the general population or any diseased subset may be predicted within the scope of the present invention, including but certainly not limited to diabetics. For example, even specifically for diabetes, all or any subset of the following standard outcomes may be predicted: any of the PROs below, functional mobility assessments for diabetic patients conducted by healthcare providers, e.g. therapists, nurses or doctors including observing for signs of instability, uneven steps, dragging feet, uneven weight-bearing, decreased step length, decreased cadence, limitation in movement of joints e.g. ankles or knees, Timed Up-and-Go (TUG) Test, Romberg Test, Four Square Step Test, Berg Balance Scale, grip strength test, or manual muscle testing, where a clinician applies resistance to, say, quadriceps or hamstrings, and asks the patient to resist, to detect indications of, say, peripheral neuropathy, assessment of ankle range of motion or deformities, Activities-Specific Balance Confidence Scale (ABC Scale), Timed 10-Meter Walk Test, or even the presence of pressure ulcers or wounds or calluses or signs of infection or discoloration or swelling on the feet or legs, Single-Leg Stance Test, Dynamic Gait Index (DGI), Sit-to-Stand Test, handheld dynamometry, Michigan Neuropathy Screening Instrument (MNSI), Semmes-Weinstein Monofilament Test, goniometry which measures range of motion, e.g. of ankles and feet, to detect diabetes-related stiffness or deformities such as Charcot foot, or Diabetes-Specific Quality of Life (DSQOL) Surveys, and fall risk (since peripheral neuropathy, which is common in diabetes, may increase fall risk). More generally, any standard assessment of clinical outcome e.g. self-report patient questionnaires or PROs, clinician assessments, lab results e.g. cancer markers, disease-specific indexes, reported falls, test results of any kind (say: intra-ocular pressure or other tests routinely done during certain routine clinic checkups) may be among the standard outcome/s predicted by the system herein.

More generally, automated prediction of standard outcomes may include generating time-stamped feature vectors for end-users in the system, each time-stamped feature vector for a given end-user Joe on a given date/time such as 3 Oct. 2024 including data points in the system for Joe (e.g. gait parameters or mobility measures of Joe's which were all measured on 3 Oct. 24, or standard outcomes such as PROs actually generated by Joe on that date, or demographic variables or clinical events), and/or interpolated data points if no measured data point is available for that date e.g. an interpolated value of a given gait parameter which may be interpolated because this parameter was measured on 3 different dates previous to 3 Oct. 24 and perhaps on date/s subsequent to 3 Oct. 24 as well, albeit not on 3 Oct. 24 itself.

Such feature vectors may be generated for plural end-users, and including for end-user David, for whom it is sought to generate an estimate of a standard outcome (say TUG), time-stamped for, say, 4 Oct. 24. Typically the plural end-users also have TUG scores in the system, which typically are not included in feature vectors generated for prediction of TUG scores. For example, user A may have a TUG score dated 1 July and user B may have a TUG score in the system dated 1 August. Then, a model (e.g. Random Forests or Gradient Boosting Machine or any suitable decision tree) may be trained to predict TUG scores (dependent variable), from feature vector/s (independent variable/s) whose time-stamps are, say, one day before that user's stored TUG score. Thus the feature vector generated for user A (to be paired with user A's 1 July's TUG score in the model's training data) may be from 30 June and the feature vector generated for user B (to be paired with user A's 1 August TUG score in the model's training data) may be from 31 July.

Alternatively, when seeking to estimate a given standard out come e.g. TUG, time-series models may be used directly on various data points (e.g. PRO's, gait parameters, clinic tests, demographic variables etc.) which are variously time-stamped, all of which typically belong to end-users in the system for whom measured TUG results, variously time-stamped, are available. In this embodiment, no interpolation between existing dates in the system to yield a feature vector of identically-time-stamped data points is typically performed and instead, forecasting occurs directly from the variously time-stamped measured data available in the system (typically for end-users for whom measured TUG results, variously time-stamped, are available in the system).

Questionnaire-type standard outcomes and standard outcomes which are patient-reported, which may be predicted e.g. using the method of FIG. 2, and may be used to enhance decision-making in any of the operations of FIG. 1, may include:
Pain rating—www.physio-pedia.com/Numeric Pain Rating Scale
The Falls Efficacy Scale—www.physio-pedia.com/Falls Efficacy Scale—International (FES-I)
Quick DASH—www.physio-pedia.com/DASH Outcome Measure
The Lower Extremity Functional Scale—www.physio-pedia.com/Lower Extremity Functional Scale (LEFS)
SF-36 or SF-12 (Short Form Health Surveys): Generic measures that evaluate functional health and well-being. www.physio-pedia.com/36-Item Short Form Survey (SF-36)
EQ-5D: A general measure used to assess health-related quality of life in patients with chronic conditions, including diabetes.
Diabetes Quality of Life (DQoL) Questionnaire: Assesses the impact of diabetes on an individual's quality of life.
Problem Areas in Diabetes (PAID) Scale: Measures diabetes-related emotional distress.
Diabetes Distress Scale (DDS): Evaluates the emotional burden associated with diabetes management.
Audit of Diabetes-Dependent Quality of Life (ADDQoL): A tool that looks at how diabetes affects various aspects of life and well-being.
Summary of Diabetes Self-Care Activities (SDSCA): A self-reported measure of diabetes self-management behaviors.
Hypoglycemia Fear Survey (HFS-II): Assesses the fear related to low blood sugar episodes.
Diabetes Treatment Satisfaction Questionnaire (DTSQ): Gauges patient satisfaction with their diabetes treatment.
CASI—The Cognitive Abilities Screening Instrument pubmed.ncbi.nlm.nih.gov/8054493/#:˜:text=The%20Cognitive%20Abi lities%20Screening%20Instrument%20(CASI)%20has%20a%20score%20range,fluency%2C%20abstraction%2C%20and%20judgment.

It is appreciated that the outcomes predicted by the system herein may be used by the system for multiple purposes e.g. all or any subset of the following (taking the Six-Minute Walk Test (6MWT) as an example outcome e.g.): scoring cardiovascular health, scoring respiratory health, e.g. an early indicator of cardiovascular or respiratory complications, which are common in diabetics, which may merit an action item on the part of the health organization that may be automatically triggered by the system, or ascertaining difficulties in performance of daily activities such as climbing stairs which may be detected, say, using a classifier which may classify simultaneously along 2 dimensions: device position (in pocket/hand etc.)×user activity (walking/running/climbing stairs etc.) e.g. as described in co-owned US patent document 20200289027 (hereby incorporated herein by reference in its entirety) which is entitled SYSTEM, METHOD AND COMPUTER PROGRAM PRODUCT FOR ASSESSMENT OF A USER'S GAIT and describing a gait monitoring system, any aspect of which may be combined with any aspect of the embodiments shown and described herein. These difficulties may be caused by (possibly undiagnosed) peripheral neuropathy and/or peripheral artery disease (PAD) which may merit an action item on the part of the health organization that may be automatically triggered by the system, such as confirming by diagnosis and/or treatment adjustment, tracking progress over time as a diabetic undergoes an exercise program or other treatment, to identify improvements or setbacks and, responsively, prompt healthcare providers to adjust the end-user's treatment plans and/or exercise routines and/or medications, identifying goals for a personalized rehabilitation or exercise program and/or establishing a baseline of an individual's physical capacity, to facilitate setting realistic personal goals for increasing activity. Uses other than assessing cardiovascular health, identifying complications, tracking improvements, and guiding personalized treatment plans are also within the scope of the invention.

It is appreciated that terminology such as “mandatory”, “required”, “need” and “must” refer to implementation choices made within the context of a particular implementation or application described herewithin for clarity, and are not intended to be limiting, since, in an alternative implementation, the same elements may be defined as not mandatory and not required. or may even be eliminated altogether.

Components described herein as software may, alternatively, be implemented wholly or partly in hardware and/or firmware, if desired, using conventional techniques, and vice-versa. Each module or component or processor may be centralized in a single physical location or physical device or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order, including simultaneous performance of suitable groups of operations, as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order, i.e., not necessarily as shown, including performing various operations in parallel or concurrently, rather than sequentially, as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform, e.g., in software, any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented, e.g., by one or more processors. The invention shown and described herein may include (a) using a computerized method to identify a solution to any of the problems or for any of the objectives described herein, the solution optionally including at least one of a decision, an action, a product, a service or any other information described herein that impacts, in a positive manner, a problem or objectives described herein; and (b) outputting the solution.

The system may, if desired, be implemented as a network—e.g., web-based system employing software, computers, routers, and telecommunications equipment, as appropriate.

Any suitable deployment may be employed to provide functionalities, e.g., software functionalities shown and described herein. For example, a server may store certain applications, for download to clients, which are executed at the client side, the server side serving only as a storehouse. Any or all functionalities, e.g., software functionalities shown and described herein, may be deployed in a cloud environment. Clients, e.g., mobile communication devices such as smartphones, may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if-then” logic described herein is intended to include embodiments in which a processor is programmed to repeatedly determine whether condition x, which is sometimes true and sometimes false, is currently true or false, and to perform y each time x is determined to be true, thereby to yield a processor which performs y at least once, typically on an “if and only if” basis, e.g., triggered only by determinations that x is true, and never by determinations that x is false.

Any determination of a state or condition described herein, and/or other data generated herein, may be harnessed for any suitable technical effect. For example, the determination may be transmitted or fed to any suitable hardware, firmware, or software module, which is known or which is described herein to have capabilities to perform a technical operation responsive to the state or condition. The technical operation may, for example, comprise changing the state or condition, or may more generally cause any outcome which is technically advantageous, given the state or condition or data, and/or may prevent at least one outcome which is disadvantageous, given the state or condition or data. Alternatively or in addition, an alert may be provided to an appropriate human operator or to an appropriate external system.

Features of the present invention, including operations which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art, and particularly, although not limited to those described in the Background section or in publications mentioned therein.

Conversely, features of the invention, including operations, which are described for brevity in the context of a single embodiment or in a certain order, may be provided separately or in any suitable sub-combination, including with features known in the art (particularly although not limited to those described in the Background section or in publications mentioned therein) or in a different order. “e.g.” is used herein in the sense of a specific example which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments, or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, Satellite including GPS, or other mobile delivery. It is appreciated that in the description and drawings shown and described herein, functionalities described or illustrated as systems and sub-units thereof may also be provided as methods and operations therewithin, and functionalities described or illustrated as methods and operations therewithin may also be provided as systems and sub-units thereof. The scale used to illustrate various elements in the drawings is merely exemplary and/or appropriate for clarity of presentation, and is not intended to be limiting.

Any suitable communication may be employed between separate units herein, e.g., wired data communication and/or in short-range radio communication with sensors such as cameras e.g., via Wifi, Bluetooth, or Zigbee.

It is appreciated that implementation via a cellular app as described herein is but an example, and, instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network, or is tethered directly or indirectly/ultimately to such a node).

Any operation or characteristic described herein may be performed by another actor outside the scope of the patent application and the description is intended to include apparatus whether hardware, firmware or software which is configured to perform, enable, or facilitate that operation or to enable, facilitate, or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry including any such computer microprocessor/s as well as in firmware or in hardware or any combination thereof.

It is appreciated that elements illustrated in more than one drawing, and/or elements in the written description, may still be combined into a single embodiment, except if otherwise specifically clarified herewithin. Any of the systems shown and described herein may be used to implement or may be combined with, any of the operations or methods shown and described herein.

It is appreciated that any features, properties, logic, modules, blocks, operations, or functionalities described herein which are, for clarity, described in the context of separate embodiments, may also be provided in combination in a single embodiment, except where the specification or general knowledge specifically indicates that certain teachings are mutually contradictory and cannot be combined. Any of the systems shown and described herein may be used to implement or may be combined with, any of the operations or methods shown and described herein.

Conversely, any modules, blocks, operations or functionalities described herein, which are, for brevity, described in the context of a single embodiment, may also be provided separately or in any suitable sub-combination, including with features known in the art. Each element, e.g., operation described herein may have all characteristics and attributes described or illustrated herein, or, according to other embodiments, may have any subset of the characteristics or attributes described herein.

It is appreciated that apps implementing any functionality herein may include a cell app, mobile app, computer app, or any other application software. Any application may be bundled with a computer and its system software, or published separately. The term “phone” and similar used herein is not intended to be limiting and may be replaced or augmented by any device having a processor, such as but not limited to a mobile telephone, or also set-top-box, TV, remote desktop computer, game console, tablet, mobile, e.g., laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node). Thus, the computing device may even be disconnected from e.g., WiFi, Bluetooth, etc., but may be tethered directly or ultimately to a networked device.

References herein to “said (or the) element x” having certain (e.g., functional or relational) limitations/characteristics, are not intended to imply that a single instance of element x is necessarily characterized by all the limitations/characteristics. Instead, “said (or the) element x” having certain (e.g. functional or relational) limitations/characteristics is intended to include both (a) an embodiment in which a single instance of element x is characterized by all of the limitations/characteristics and (b) embodiments in which plural instances of element x are provided, and each of the limitations/characteristics is satisfied by at least one instance of element x, but no single instance of element x satisfies all limitations/characteristics. For example, each time L limitations/characteristics are ascribed to “said” or “the” element X in the specification or claims (e.g. to “said processor” or “the processor”), this is intended to include an embodiment in which L instances of element X are provided, which respectively satisfy the L limitations/characteristics, each of the L instances of element X satisfying an individual one of the L limitations/characteristics. The plural instances of element x need not be identical. For example, if element x is a hardware processor, there may be different instances of x, each programmed for different functions and/or having different hardware configurations (e.g., there may be 3 instances of x: two Intel processors of different models, and one AMD processor).

1. A health management system comprising:
a personnel allocation processor for allocating personnel to conduct home or clinic sessions with diabetes patients known to the system; and
a gait analysis processor receiving IMU data from cellphones worn by the diabetes patients and providing the personnel allocation processor with indications of a subset of the diabetes patients whose IMU data indicates new deterioration;
and wherein allocation of personnel by the personnel allocation processor at least partly favors diabetes patients falling within the subset over diabetes patients who do not fall within the subset;
wherein the personnel allocation processor may comprise doctors' office software for managing appointments, such software having priority logic accepting prioritization via a machine interface.

2. A health management method comprising:
using gait analysis of at least one end-user's motor behavior over a time period preceding or culminating at time-point T to generate at least one estimate of at least one standard outcome at time T, for the at least one end-user; and
automatically adjusting healthcare for at least one end-user after time-point T, based at least on said estimate.

3. A system according to claim 1 wherein prioritization may comprise patients within the subset being defined as urgent and at least patients not falling within the subset being defined as routine rather than urgent.

4. A system according to claim 1 wherein the system contacts patients within the subset to request and upload an image of the patient's foot, and wherein the image is automatically image-processed to identify discoloration each time the new deterioration suggests an increase in risk of gangrene.

5. A system according to claim 1 wherein the system contacts patients within the subset to request and upload an image of the patient's foot, and wherein the image is automatically image-processed to identify changes in foot shape or alignment relative to previous images in the system and/or relative to population norms each time the new deterioration suggests an increase in risk that nerve damage has led to a weakening of bones in the foot.

6. A system according to claim 1 wherein the gait analysis processor separately classifies peripheral artery disease which leads to leg pain, and peripheral artery disease which leads to numbness.

7. A system according to claim 1 wherein the gait analysis processor is trained to predict at least one direct testing result, such as ankle brachial index test, or foot imaging tests evaluating arterial blockage, such as ultrasound or angiography.

8. A system according to claim 1 wherein the gait analysis processor has a machine interface for diabetes patients' medical records, and wherein symptoms such as peripheral artery disease are automatically added to patients' medical records responsive to classification of a patient as suffering from said symptoms by said gait analysis processor with an option for human override and/or with an indication in the medical record that the symptom has been added to the medical record using machine intelligence.

9. A system according to claim 1 wherein the gait analysis processor, responsive to identification of diabetic neuropathy by the system, is configured to schedule evaluations of neuropathic arthropathy, where each evaluation includes prompting the patient identified as having diabetic neuropathy to upload an image, and automatically processing the image to identify foot shape changes.

10. A computer program product, comprising a typically non-transitory computer-usable or -readable medium e.g. non-transitory computer-usable or -readable storage medium, having a computer readable program code embodied therein, said computer readable program code adapted to be executed to implement a health management method comprising:
using gait analysis of at least one end-user's motor behavior over a time period preceding or culminating at time-point T to generate at least one estimate of at least one standard outcome at time T, for the at least one end-user; and
automatically adjusting healthcare for at least one end-user after time-point T, based at least on said estimate.
